(19) United States
(12) Patent Application Publication
(10) Pub. No.: US 2025/0319307 A1
(43) Pub. Date: Oct. 16, 2025

(54) NEUROSTIMULATION SYSTEMS USING SENSE AND OUTCOMES DATA

(71) Applicant: Boston Scientific Neuromodulation Corporation, Valencia, CA (US)

(72) Inventors: Lisa Denise Moore, Glendale, CA (US); G. Karl Steinke, Valencia, CA (US)

(21) Appl. No.: 19/171,993

(22) Filed: Apr. 7, 2025

Related U.S. Application Data
(60) Provisional application No. 63/634,116, filed on Apr. 15, 2024.

(51) Int. Cl. A61N 1/36; A61N 1/08

(52) U.S. Cl. CPC A61N 1/36; A61N 1/08

(57) ABSTRACT

A system may include a neurostimulator and a processing system configured to provide a stimulation effects map by testing stimulation parameter sets from a plurality of available stimulation parameter sets, acquiring clinical effect data indicative of a patient response to electrical energy delivered to the tissue using at least a first subset of the tested stimulation parameter sets, acquiring sensed data indicative of a sensed response to the electrical energy delivered to the tissue using at least a second subset of the tested stimulation parameter sets, and evaluating parameter sets from the plurality of untested stimulation parameter sets including for each of the evaluated parameter sets, determining an estimated response by estimating at least one of the patient response or the sensed response to the electrical energy using the acquired clinical effect data and the acquired sensed data. A stimulation parameter set may be chosen based on the map.

### CLAIM OF PRIORITY

This application claims the benefit of U.S. Provisional Application No. 63/634,116, filed on Apr. 15, 2024, which is hereby incorporated by reference in its entirety.

### TECHNICAL FIELD

This document relates generally to medical devices, and more particularly, to systems, devices and methods programming a neurostimulation system using sense data.

### BACKGROUND

Medical devices may include therapy-delivery devices configured to deliver a therapy to a patient and/or monitors configured to monitor a patient condition via user input and/or sensor(s). Examples include wearable devices such as but not limited to, transcutaneous electrical neural stimulators (TENS), external or implantable stimulation devices such as but not limited to spinal cord stimulators (SCS) to treat chronic pain, cortical and Deep Brain Stimulators (DBS) to treat motor and psychological disorders, Peripheral Nerve Stimulation (PNS), Functional Electrical Stimulation (FES), and other neural stimulators to treat urinary incontinence, sleep apnea, shoulder subluxation, and the like.

A therapy device may be configured or programmed to treat a condition. Thus, by way of example and not limitation, a DBS system may be configured to treat motor disorders such as, but not limited to, tremor, bradykinesia, and dyskinesia associated with Parkinson's Disease (PD). In another nonlimiting example, a stimulation device, such as neurostimulation device (e.g., DBS, SCS, PNS or TENS), may be configured to treat pain. Settings of the therapy device, including stimulation parameters, may be programmed based on observed clinical effects so that the therapy provides desirable intended effects (e.g., reduced tremor, bradykinesia, and dyskinesia for a PD therapy, desirable pain relief or paresthesia coverage for a pain therapy) while avoiding undesirable side effects.

Programming an adjustment is an ongoing challenge in neurostimulation. This disclosure provides an improved system and process for programming the therapy device using measured and estimated sense data.

### SUMMARY

An example (e.g., “Example 1”) of a system may include a neurostimulator configured to use stimulation parameters to deliver electrical energy to tissue in a patient, and a processing system configured to provide a stimulation effects map that maps stimulation effects for different stimulation parameters. The stimulation effects map may be provided by testing stimulation parameter sets from a plurality of available stimulation parameter sets by controlling the neurostimulator for each of the tested stimulation parameter sets to deliver the electrical energy using the corresponding stimulation parameter set. The plurality of available stimulation parameter sets may include the tested stimulation parameter sets and a plurality of untested stimulation parameter sets. The stimulation effects map may further be provided by acquiring clinical effect data indicative of a patient response to the electrical energy delivered to the tissue using at least a first subset of the tested stimulation parameter sets, acquiring sensed data indicative of a sensed response to the electrical energy delivered to the tissue using at least a second subset of the tested stimulation parameter sets, and evaluating parameter sets from the plurality of untested stimulation parameter sets to provide evaluated parameter sets, including for each of the evaluated parameter sets, determining an estimated response by estimating at least one of the patient response or the sensed response to the electrical energy using the acquired clinical effect data and the acquired sensed data. The stimulation effects map may include the acquired clinical effect data, the acquired sensed data, and the estimated responses for the evaluated parameter sets. Optionally, the processing system may be configured to format and display the stimulation effects map to the user.

In Example 2, the subject matter of Example 1 may optionally be configured such that the processing system is configured to: based on the stimulation effects map including at least one or more of the estimated responses, choose a stimulation parameter set from the plurality of available stimulation parameter sets as a chosen stimulation parameter set to be tested; and control the neurostimulator to deliver electrical energy using the chosen stimulation parameter set. Optionally, the stimulation parameter set may be evaluated, scored and/or selected by the processing system. Optionally, the processing system may recommend or highlight to the user the parameter set to be tested.

In Example 3, the subject matter of Example 2 may optionally be configured such that the chosen parameter set is selected from one of the evaluated parameter sets, and the processing system is configured to acquire a tested response, including at least one of clinical effect data or sensed data, when the electrical energy is delivered using the chosen parameter set compare the acquired tested response to the corresponding one of the estimated responses to provide comparison data, and use the comparison data to update a model used to determine the estimated response.

In Example 4, the subject matter of any one or more of Examples 1-3 may optionally be configured to determine the estimated response by interpolating or extrapolating, including by line or surface fittings of the tested stimulation sets.

In Example 5, the subject matter of any one or more of Examples 1-4 may optionally be configured such that the processing system is configured to determine the estimated response using knowledge of an expected topology of a search, parameter or other space corresponding to the plurality of available stimulation sets, where the expected topology can include slopes and orientations, peaks, valleys, cliffs or plateaus in the search space.

In Example 6, the subject matter of any one or more of Examples 1-4 may optionally be configured such that the processing system is configured to use machine learning to estimate at least one of the patient response or the sensed response to the electrical energy using the acquired clinical effect data and the acquire sensed data.

In Example 7, the subject matter of Example 6 may optionally be configured such that the machine learning is used to analyze data from a current patient, data across multiple patients other than the current patient, or data across multiple patients including the current patient to estimate the at least one of the patient response or the sensed response.

In Example 8, the subject matter of any one or more of Examples 1-7 may optionally be configured to further include at least one of: a sensor configured to sense an electrical response from the patient to the electrical energy delivered to the tissue, where the sensed data is indicative of the sensed electrical response; or a physical sensor configured to sense a physical characteristic for the patient, where the sensed data is indicative of the physical characteristic for the patient. For example, the physical sensor may be configured for use in detecting or determining rigidity, stiffness, muscle tension, or movement. For example, the physical sensors may include internal physical sensors or external physical sensors.

In Example 9, the subject matter of any one or more of Examples 1-8 may optionally be configured such that the processing system includes a user interface configured to receive a user input indicative of the patient response to the electrical energy delivered to the tissue, where the user input is indicative of at least one of: one or more side effects to the electrical energy delivered to the tissue; or whether and to what extent the electrical energy delivered to the tissue is therapeutically effective.

In Example 10, the subject matter of any one or more of Examples 1-9 may optionally be configured such that each of the plurality of available stimulation parameter sets includes an electrode configuration and a stimulation waveform configuration.

In Example 11, the subject matter of any one or more of Examples 1-10 may optionally be configured such that both the clinical effect data and the sensed data are associated with at least two stimulation parameters in the plurality of available stimulation parameter sets.

In Example 12, the subject matter of Example 11 may optionally be configured such that both the clinical effect data and the sensed data are associated with a stimulation location and a stimulation amplitude.

In Example 13, the subject matter of Example 12 may optionally be configured such that both the clinical effect data and the sensed data are further associated with at least one of: a stimulation frequency, a stimulation pulse width, a stimulation location and a stimulation amplitude.

In Example 14, the subject matter of any one or more of Examples 1-13 may optionally be configured such that the sensed data includes features of a sensed signal, and the processing system is configured to determine a difference between the estimated sensed response and the sensed signal and use the determined difference to choose the stimulation parameter set as the chosen stimulation parameter set to be tested.

In Example 15, the subject matter of any one or more of Examples 1-14 may optionally be configured such that the sensed data includes features of a sensed signal, and the processing system is configured to determine a difference between the sensed signal and the estimated sensed response, determine the estimated patient response based on the determined difference and display the estimated patient response on a user interface.

Example 16 includes subject matter (such as a method, means for performing acts, machine readable medium including instructions that when performed by a machine cause the machine to perform acts, or an apparatus to perform). The subject matter may include using a neurostimulator to use stimulation parameters to deliver electrical energy to tissue in a patient and using processing system to provide a stimulation effects map that maps stimulation effects for different stimulation parameters. The map may be provided by testing stimulation parameter sets from a plurality of available stimulation parameter sets by controlling the neurostimulator for each of the tested stimulation parameter sets to deliver the electrical energy using the corresponding stimulation parameter set. The plurality of available stimulation parameter sets may include the tested stimulation parameter sets and a plurality of untested stimulation parameter sets. The map may be provided by acquiring clinical effect data indicative of a patient response to the electrical energy delivered to the tissue using at least a first subset of the tested stimulation parameter sets, acquiring sensed data indicative of a sensed response to the electrical energy delivered to the tissue using at least a second subset of the tested stimulation parameter sets, and evaluating parameter sets from the plurality of untested stimulation parameter sets to provide evaluated parameter sets, including for each of the evaluated parameter sets, determine an estimated response by estimating at least one of the patient response or the sensed response to the electrical energy using the acquired clinical effect data and the acquired sensed data. The stimulation effects map may include the acquired clinical effect data, the acquired sensed data, and the estimated responses for the evaluated parameter sets. Optionally, the processing system may be configured to format and display the stimulation effects map to the user.

In Example 17, the subject matter of Example 16 may optionally be configured to further include using the processing system to choose, based on at least one or more of the estimated responses, a stimulation parameter set from the plurality of available stimulation parameter sets as a chosen stimulation parameter set to be tested and control the neurostimulator to deliver electrical energy using the chosen stimulation parameter set. Optionally, the stimulation parameter set may be evaluated, scored and/or selected by the processing system. Optionally, the processing system may recommend or highlight to the user the parameter set to be tested.

In Example 18, the subject matter of any one or more of Examples 16-17 may optionally be configured such that the chosen parameter set is selected from one of the evaluated parameter sets, and the subject matter further includes acquiring a tested response, including at least one of clinical effect data or sense data, when the electrical energy is delivered using the chosen parameter set, comparing the acquired tested response to the corresponding one of the estimated responses to provide comparison data, and using the comparison data to update a model used to determine the estimated response.

In Example 19, the subject matter of any one or more of Examples 16-18 may optionally be configured such that the estimated response is determined by interpolating or extrapolating, including by line or surface fittings of the tested stimulation sets.

In Example 20, the subject matter of any one or more of Examples 16-19 may optionally be configured such that the estimated response is determined using knowledge of an expected topology of a search, parameter or other space corresponding to the plurality of available stimulation sets, where the expected topology can include slopes and orientations, peaks, valleys, cliffs or plateaus in the search space.

In Example 21, the subject matter of any one or more of Examples 16-20 may optionally be configured to further include using machine learning for estimating at least one of the patient response or the sensed response to the electrical energy using the acquired clinical effect data and the acquire sensed data.

In Example 22, the subject matter of Example 21 may optionally be configured such that the machine learning analyzes data from a current patient, analyzes data across multiple patients other than the current patient, or analyzes data across multiple patients including the current patient to estimate the at least one of the patient response or the sensed response.

In Example 23, the subject matter of any one or more of Examples 16-22 may optionally be configured to further include at least one of: sensing an electrical response from the patient to the electrical energy delivered to the tissue, where the sensed data is indicative of the sensed electrical response; or sensing a physical characteristic for the patient, where the sensed data is indicative of the physical characteristic for the patient. For example, the physical sensor may be configured for use in detecting or determining rigidity, stiffness, muscle tension, or movement. For example, the physical sensors may include internal physical sensors or external physical sensors.

In Example 24, the subject matter of any one or more of Examples 16-23 may optionally be configured to further include receiving a user input indicative of the patient response to the electrical energy delivered to the tissue, where the user input is indicative of at least one of one or more side effects to the electrical energy delivered to the tissue or whether and to what extent the electrical energy delivered to the tissue is therapeutically effective.

In Example 25, the subject matter of any one or more of Examples 16-24 may optionally be configured such that each of the plurality of available stimulation parameter sets includes an electrode configuration and a stimulation waveform configuration.

In Example 26, the subject matter of any one or more of Examples 16-25 may optionally be configured such that both the clinical effect data and the sensed data are associated with at least two stimulation parameters in the plurality of available stimulation parameter sets.

In Example 27, the subject matter of Example 26 may optionally be configured such both the clinical effect data and the sensed data are associated with a stimulation location and a stimulation amplitude.

In Example 28, the subject matter of Example 27 may optionally be configured such that both the clinical effect data and the sensed data are further associated with at least one of: a stimulation frequency, a stimulation pulse width, a stimulation location and a stimulation amplitude.

In Example 29, the subject matter of any one or more of Examples 16-28 may optionally be configured such that the sensed data includes features of a sensed signal, and the subject matter further includes determining a difference between the estimated sensed response and the sensed signal and using the determined difference to choose the stimulation parameter set as the chosen stimulation parameter set to be tested.

In Example 30, the subject matter of any one or more of Examples 16-29 may optionally be configured such that the sensed data includes features of a sensed signal, and the subject matter further includes determining a difference between the sensed signal and the estimated sensed response, determining the estimated patient response based on the determined difference and displaying the estimated patient response on a user interface.

Example 31 includes subject matter that includes non-transitory machine-readable medium including instructions, which when executed by a machine, cause the machine to perform a method. The method may include, by way of example and not limitation, any of the subject matter for one or more of Examples 16-30. The machine-readable medium may include instructions operable to configure an electronic device to perform methods as described in the above examples. An implementation of such methods may include code, such as microcode, assembly language code, a higher-level language code, or the like. Such code may include computer readable instructions for performing various methods. The code may form portions of computer program products. Further, in an example, the code may be tangibly stored on one or more volatile, non-transitory, or non-volatile tangible computer-readable media, such as during execution or at other times. Examples of these tangible computer-readable media may include, but are not limited to, hard disks, removable magnetic disks or cassettes, removable optical disks (e.g., compact disks and digital video disks), memory cards or sticks, random access memories (RAMs), read only memories (ROMs), and the like. The term “machine-readable medium” is intended to include at least one machine-readable medium (e.g., two or more media which may be of the same type of media (such as but not limited to different nonvolatile semiconductor memory arrays) or different type of media (such as but not limited to a hard disk and a non-volatile semiconductor memory array). Furthermore, the term “machine” may include at least one processor, including one processor to implement all of the instructions, at least two processors where one processor operates on some of the instructions and other processor(s) operate on other instructions, or at least two processors where each processor is capable of operating on the same instructions. Thus, for example, distributed systems or systems with shared resources are contemplated.

This Summary is an overview of some of the teachings of the present application and not intended to be an exclusive or exhaustive treatment of the present subject matter. Further details about the present subject matter are found in the detailed description and appended claims. Other aspects of the disclosure will be apparent to persons skilled in the art upon reading and understanding the following detailed description and viewing the drawings that form a part thereof, each of which are not to be taken in a limiting sense. The scope of the present disclosure is defined by the appended claims and their legal equivalents.

### BRIEF DESCRIPTION OF THE DRAWINGS

Various examples are illustrated by way of example in the figures of the accompanying drawings. Such examples are demonstrative and not intended to be exhaustive or exclusive examples of the present subject matter.

FIG. 1 illustrates an example of an electrical stimulation system that may be used to deliver deep brain stimulation (DBS).

FIG. 2 illustrates an example of an implantable pulse generator (IPG) that may be used in a DBS system.

FIGS. 3A-3B illustrate examples of leads that may be coupled to an IPG to deliver electrostimulation such as DBS.

FIG. 4 illustrates an example of a computing device for programming or controlling the operation of an electrostimulation system.

FIG. 5 illustrates an example of a stimulation parameter control system and a part of the environment in which it may operate.

FIG. 6 illustrates, by way of example, an example of an electrical therapy-delivery system.

FIG. 7 illustrates, by way of example and not limitation, an implantable electrical therapy-delivery system.

FIG. 8 illustrates a therapy being delivered according to a parameter set.

FIG. 9 illustrates a therapy space, which includes different parameter sets potentially available for delivering the therapy.

FIG. 10 illustrates, by way of example and not limitation, a two-dimensional plot of location and amplitude for measured and estimated outcomes (e.g., clinical effects) and sensed and estimated data.

FIG. 11 illustrates, by way of example and not limitation, a stimulation effects map having multidimension relationships between a stimulation parameter set (illustrated as including N parameter(s)) and each of a patient response and sensed data.

FIG. 12 illustrates, by way of example and not limitation, available stimulation parameter sets.

FIG. 13 illustrates, by way of example and not limitation, a method for providing a stimulation effects map which may be used to choose a parameter set to be tested or used for therapy.

FIG. 14 illustrates, by way of example and not limitation, a method for choosing a parameter set to be tested based on a difference between estimated response(s) and acquired response(s).

FIG. 15 illustrates, by way of example and not limitation, a method for displaying an estimated patient response determined based on a difference between sensed and estimated sense data.

FIG. 16 illustrates, by way of example and not limitation, a method for updating estimated responses.

### DETAILED DESCRIPTION

The following detailed description of the present subject matter refers to the accompanying drawings which show, by way of illustration, specific aspects and embodiments in which the present subject matter may be practiced. These embodiments are described in sufficient detail to enable those skilled in the art to practice the present subject matter. Other embodiments may be utilized and structural, logical, and electrical changes may be made without departing from the scope of the present subject matter. References to “an”, “one”, or “various” embodiments in this disclosure are not necessarily to the same embodiment, and such references contemplate more than one embodiment. The following detailed description is, therefore, not to be taken in a limiting sense, and the scope is defined only by the appended claims, along with the full scope of legal equivalents to which such claims are entitled.

Disclosed herein, among other things, is a system and method for mapping neurostimulation effects using both patient outcomes and sensed signals at multiple settings (e.g., locations and amplitudes) to predict patient responses (e.g., patient outcome and/or sensed signal characteristics) at untested locations and/or amplitudes. Understanding an anticipated magnitude of change in therapy, side effect outcome can assist in programming. For example, a system may be configured to implement an algorithm to select or recommend preferred or next stimulation settings for the neural stimulator based on an expected magnitude of change between parameter sets, which may be used. A system may be configured to display patient outcomes and sensed data for tested stimulation parameter sets as well as predicted patient responses (e.g., patient outcome and/or sensed signal characteristics) for some untested stimulation parameter sets.

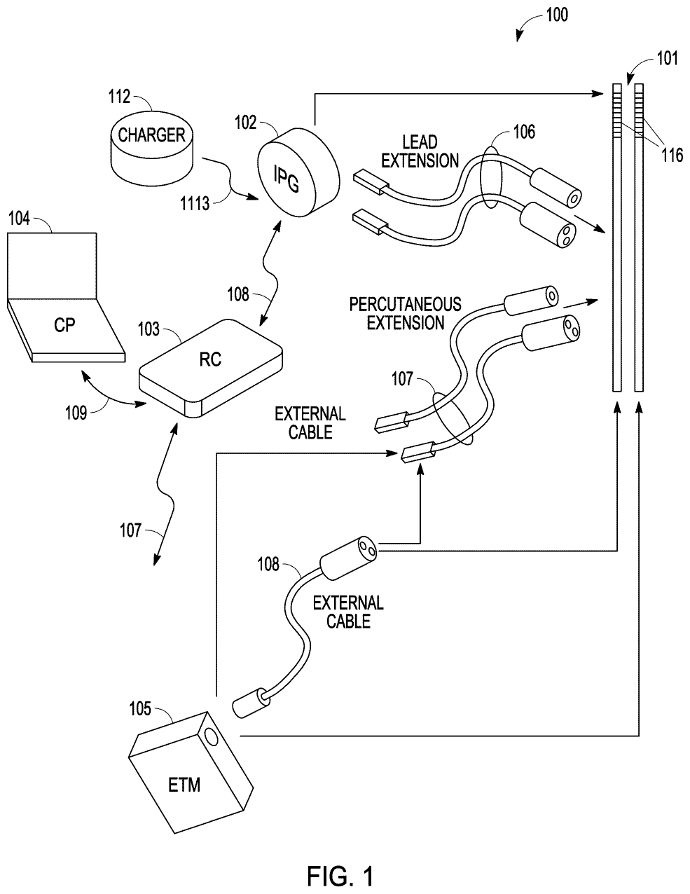

FIG. 1 illustrates, by way of example and not limitation, an electrical stimulation system 100, which may be used to deliver DBS. The electrical stimulation system 100 may generally include a one or more (illustrated as two) of implantable neurostimulation leads 101, a waveform generator such as an implantable pulse generator (IPG) 102, an external remote controller (RC) 103, a clinician programmer (CP) 104, and an external trial modulator (ETM) 105. The IPG 102 may be physically connected via one or more percutaneous lead extensions 106 to the neurostimulation lead(s) 101, which carry a plurality of electrodes 116. The electrodes, when implanted in a patient, form an electrode arrangement. As illustrated, the neurostimulation leads 101 may be percutaneous leads with the electrodes arranged in-line along the neurostimulation leads or about a circumference of the neurostimulation leads. Any suitable number of neurostimulation leads can be provided, including only one, as long as the number of electrodes is greater than two (including the IPG case function as a case electrode) to allow for lateral steering of the current. Alternatively, a surgical paddle lead can be used in place of one or more of the percutaneous leads. The IPG 102 includes pulse generation circuitry that delivers electrical stimulation energy in the form of a pulsed electrical waveform (i.e., a temporal series of electrical pulses) to the electrodes in accordance with a set of stimulation parameters.

The ETM 105 may also be physically connected via the percutaneous lead extensions 107 and external cable 108 to the neurostimulation lead(s) 101. The ETM 105 may have similar pulse generation circuitry as the IPG 102 to deliver electrical stimulation energy to the electrodes in accordance with a set of stimulation parameters. A programming process may be used to test different parameter sets. The ETM 105 is a non-implantable device that may be used on a trial basis after the neurostimulation leads 101 have been implanted and prior to implantation of the IPG 102, to test the responsiveness of the stimulation that is to be provided. Functions described herein with respect to the IPG 102 can likewise be performed with respect to the ETM 105.

The RC 103 may be used to telemetrically control the ETM 105 via a bi-directional RF communications link 109. The RC 103 may be used to telemetrically control the IPG 102 via a bi-directional RF communications link 110. Such control allows the IPG 102 to be turned on or off and to be programmed with different stimulation parameter sets. The IPG 102 may also be operated to modify the programmed stimulation parameters to actively control the characteristics of the electrical stimulation energy output by the IPG 102. A clinician may use the CP 104 to program stimulation parameters into the IPG 102 and ETM 105 in the operating room and in follow-up sessions.

The CP 104 may indirectly communicate with the IPG 102 or ETM 105, through the RC 103, via an IR communications link 111 or another link. The CP 104 may directly communicate with the IPG 102 or ETM 105 via an RF communications link or other link (not shown). The clinician detailed stimulation parameters provided by the CP 104 may also be used to program the RC 103, so that the stimulation parameters can be subsequently modified by operation of the RC 103 in a stand-alone mode (i.e., without the assistance of the CP 104). Various devices may function as the CP 104. Such devices may include portable devices such as a lap-top personal computer, mini-computer, personal digital assistant (PDA), tablets, phones, or a remote control (RC) with expanded functionality. Thus, the programming methodologies can be performed by executing software instructions contained within the CP 104. Alternatively, such programming methodologies can be performed using firmware or hardware. In any event, the CP 104 may actively control the characteristics of the electrical stimulation generated by the IPG 102 to allow the desired parameters to be determined based on patient feedback or other feedback and for subsequently programming the IPG 102 with the desired stimulation parameters. To allow the user to perform these functions, the CP 104 may include user input device (e.g., a mouse and a keyboard), and a programming display screen housed in a case. In addition to, or in lieu of, the mouse, other directional programming devices may be used, such as a trackball, touchpad, joystick, touch screens or directional keys included as part of the keys associated with the keyboard. An external device (e.g. CP) may be programmed to provide display screen(s) that allow the clinician to, among other functions, select or enter patient profile information (e.g., name, birth date, patient identification, physician, diagnosis, and address), enter procedure information (e.g., programming/follow-up, implant trial system, implant IPG, implant IPG and lead(s), replace IPG, replace IPG and leads, replace or revise leads, explant, etc.), generate a pain map of the patient, define the configuration and orientation of the leads, initiate and control the electrical stimulation energy output by the neurostimulation leads, and select and program the IPG with stimulation parameters, including electrode selection, in both a surgical setting and a clinical setting. The external device(s) (e.g., CP and/or RC) may be configured to communicate with other device(s), including local device(s) and/or remote device(s). For example, wired and/or wireless communication may be used to communicate between or among the devices.

An external charger 112 may be a portable device used to transcutaneous charge the IPG 102 via a wireless link such as an inductive link 113. Once the IPG 102 has been programmed, and its power source has been charged by the external charger or otherwise replenished, the IPG 102 may function as programmed without the RC 103 or CP 104 being present.

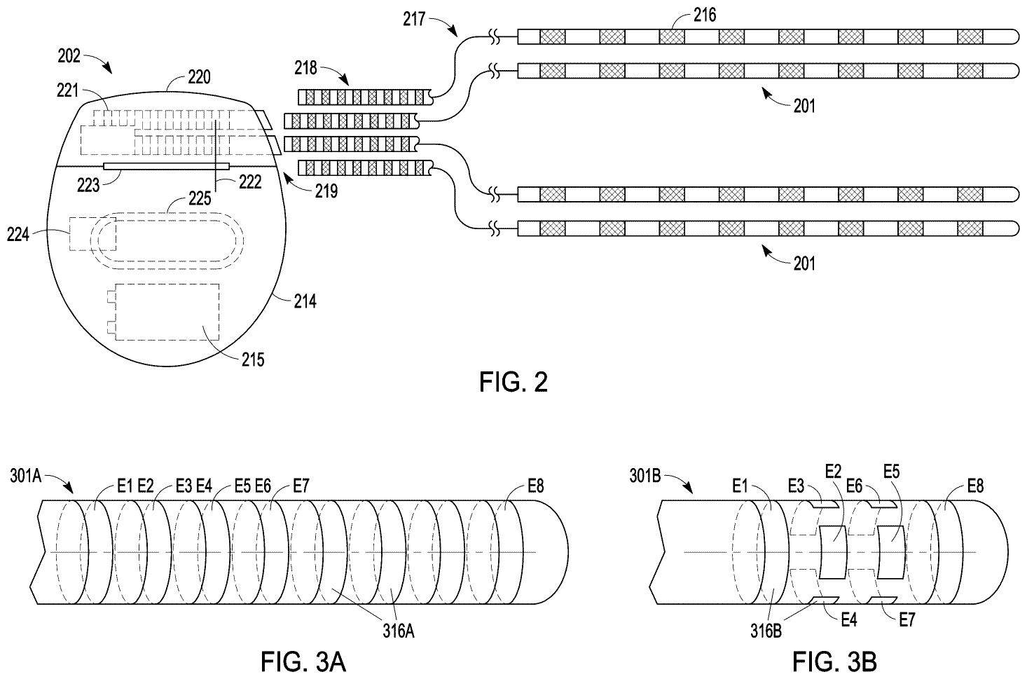

FIG. 2 illustrates, by way of example and not limitation, an IPG 202 in a DBS system. The IPG 202, which is an example of the IPG 102 of the electrical stimulation system 100 as illustrated in FIG. 1, may include a biocompatible device case 214 that holds the circuitry and a battery 215 for providing power for the IPG 202 to function, although the IPG 202 may also lack a battery and may be wirelessly powered by an external source. The IPG 202 may be coupled to one or more leads, such as leads 201 as illustrated herein. The leads 201 may each include a plurality of electrodes 216 for delivering electrostimulation energy, recording electrical signals, or both. In some examples, the leads 201 may be rotatable so that the electrodes 216 may be aligned with the target neurons after the neurons have been located such as based on the recorded signals. The electrodes 216 may include one or more ring electrodes, and/or one or more sets of segmented electrodes (or any other combination of electrodes), examples of which are discussed below with reference to FIGS. 3A and 3B.

The leads 201 may be implanted near or within the desired portion of the body to be stimulated. In an example of operations for DBS, access to the desired position in the brain may be accomplished by drilling a hole in the patient's skull or cranium with a cranial drill (commonly referred to as a burr), and coagulating and incising the dura mater, or brain covering. A lead may then be inserted into the cranium and brain tissue with the assistance of a stylet (not shown). The lead may be guided to the target location within the brain using, for example, a stereotactic frame and a microdrive motor system. In some examples, the microdrive motor system may be fully or partially automatic. The microdrive motor system may be configured to perform actions such as inserting, advancing, rotating, or retracing the lead.

Lead wires 217 within the leads may be coupled to the electrodes 216 and to proximal contacts 218 insertable into lead connectors 219 fixed in a header 220 on the IPG 202, which header may comprise an epoxy for example. Alternatively, the proximal contacts 218 may connect to lead extensions (not shown) which are in turn inserted into the lead connectors 219. Once inserted, the proximal contacts 218 connect to header contacts 221 within the lead connectors 219, which are in turn coupled by feedthrough pins 222 through a case feedthrough 223 to stimulation circuitry 224 within the case 214. The type and number of leads, and the number of electrodes, in an IPG is application specific and therefore can vary.

The IPG 202 may include an antenna 225 allowing it to communicate bi-directionally with a number of external devices. The antenna 225 may be a conductive coil within the case 214, although the coil of the antenna 225 may also appear in the header 220. When the antenna 225 is configured as a coil, communication with external devices may occur using near-field magnetic induction. The IPG 202 may also include a Radio-Frequency (RF) antenna. The RF antenna may comprise a patch, slot, or wire, and may operate as a monopole or dipole, and preferably communicates using far-field electromagnetic waves, and may operate in accordance with any number of known RF communication standards, such as Bluetooth, Zigbee, WiFi, MICS, and the like.

In a DBS application, as is useful in the treatment of tremor in Parkinson's disease for example, the IPG 202 is typically implanted under the patient's clavicle (collarbone). The leads 201 (which may be extended by lead extensions, not shown) may be tunneled through and under the neck and the scalp, with the electrodes 216 implanted through holes drilled in the skull and positioned for example in the subthalamic nucleus (STN) and the pedunculopontine nucleus (PPN) in each brain hemisphere. The IPG 202 may also be implanted underneath the scalp closer to the location of the electrodes' implantation. The leads 201, or the extensions, may be integrated with and permanently connected to the IPG 202 in other solutions.

Stimulation in IPG 202 is typically provided by pulses each of which may include one phase or multiple phases. For example, a monopolar stimulation current may be delivered between a lead-based electrode (e.g., one of the electrodes 216) and a case electrode. A bipolar stimulation current may be delivered between two lead-based electrodes (e.g., two of the electrodes 216). Stimulation parameters typically include current amplitude (or voltage amplitude), frequency, pulse width of the pulses or of its individual phases; electrodes selected to provide the stimulation; polarity of such selected electrodes, i.e., whether they act as anodes that source current to the tissue, or cathodes that sink current from the tissue. Each of the electrodes may either be used (an active electrode) or unused (OFF). When the electrode is used, the electrode may be used as an anode or cathode and carry anodic or cathodic current. The anodic energy contributions may be distributed across more than one anode and the cathodic energy contributions may be distributed across more than one cathode (e.g., electrode fractionalization). Thus, by way of example and not limitation, one electrode may be programmed to provide all (100%) of the anodic energy, and four electrodes may be programmed to provide fractions (e.g., 25%, 25%, 25%, 25%; or 10%, 20%, 30% and 40%) of the total cathodic energy. In some instances, an electrode might be an anode for a period of time and a cathode for a period of time. These and possibly other stimulation parameters taken together comprise a stimulation program that the stimulation circuitry 224 in the IPG 202 may execute to provide therapeutic stimulation to a patient.

In some examples, a measurement device coupled to the muscles or other tissue stimulated by the target neurons, or a unit responsive to the patient or clinician, may be coupled to the IPG 202 or microdrive motor system. The measurement device, user, or clinician may indicate a response by the target muscles or other tissue to the stimulation or recording electrode(s) to further identify the target neurons and facilitate positioning of the stimulation electrode(s). For example, if the target neurons are directed to a muscle experiencing tremors, a measurement device may be used to observe the muscle and indicate changes in, for example, tremor frequency or amplitude in response to stimulation of neurons. Alternatively, the patient or clinician may observe the muscle and provide feedback.

FIGS. 3A-3B illustrate, by way of example and not limitation, leads that may be coupled to the IPG to deliver electrostimulation such as DBS. FIG. 3A shows a lead 301A with electrodes 316A disposed at least partially about a circumference of the lead 301A. The electrodes 316A may be located along a distal end portion of the lead. As illustrated herein, the electrodes 316A are ring electrodes that span 360 degrees about a circumference of the lead 301. A ring electrode allows current to project equally in every direction from the position of the electrode, and typically does not enable stimulus current to be directed from only a particular angular position or a limited angular range around of the lead. A lead which includes only ring electrodes may be referred to as a non-directional lead.

FIG. 3B shows a lead 301B with electrodes 316B including ring electrodes such as E1 at a proximal end and E8 at the distal end. Additionally, the lead 301 also include a plurality of segmented electrodes (also known as split-ring electrodes). For example, a set of segmented electrodes E2, E3, and E4 are around the circumference at a longitudinal position, each spanning less than 360 degrees around the lead axis. In an example, each of electrodes E2, E3, and E4 spans 90 degrees, with each being separated from the others by gaps of 30 degrees. Another set of segmented electrodes E5, E6, and E7 are located around the circumference at another longitudinal position different from the segmented electrodes E2, E3 and E4. Segmented electrodes such as E2-E7 may direct stimulus current to a selected angular range around the lead.

Segmented electrodes may typically provide superior current steering than ring electrodes because target structures in DBS or other stimulation are not typically symmetric about the axis of the distal electrode array. Instead, a target may be located on one side of a plane running through the axis of the lead. Through the use of a radially segmented electrode array, current steering may be performed not only along a length of the lead but also around a circumference of the lead. This provides precise three-dimensional targeting and delivery of the current stimulus to neural target tissue, while potentially avoiding stimulation of other tissue. In some examples, segmented electrodes may be together with ring electrodes. A lead which includes at least one or more segmented electrodes may be referred to as a directional lead. In an example, all electrodes on a directional lead may be segmented electrodes. In another example, there may be different numbers of segmented electrodes at different longitudinal positions.

Segmented electrodes may be grouped into sets of segmented electrodes, where each set is disposed around a circumference at a particular longitudinal location of the directional lead. The directional lead may have any number of segmented electrodes in a given set of segmented electrodes. By way of example and not limitation, a given set may include any number between two to sixteen segmented electrodes. In an example, all sets of segmented electrodes may contain the same number of segmented electrodes. In another example, one set of the segmented electrodes may include a different number of electrodes than at least one other set of segmented electrodes.

The segmented electrodes may vary in size and shape. In some examples, the segmented electrodes are all of the same size, shape, diameter, width or area or any combination thereof. In some examples, the segmented electrodes of each circumferential set (or even all segmented electrodes disposed on the lead) may be identical in size and shape. The sets of segmented electrodes may be positioned in irregular or regular intervals along a length the lead.

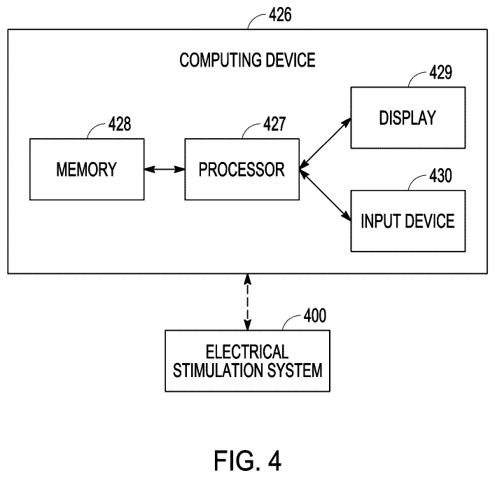

FIG. 4 illustrates, by way of example and not limitation, a computing device 426 for programming or controlling the operation of an electrical stimulation system 400. The computing device 426 may include a processor 427, a memory 428, a display 429, and an input device 430. Optionally, the computing device 426 may be separate from and communicatively coupled to the electrical stimulation system 400, such as system 100 in FIG. 1 Alternatively, the computing device 426 may be integrated with the electrical stimulation system 100, such as part of the IPG 102, RC 103, CP 104, or ETM 105 illustrated in FIG. 1.

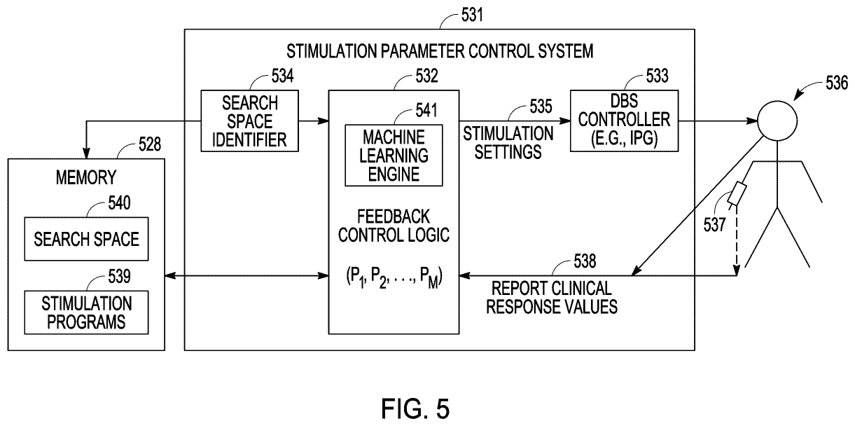

The computing device 426, also referred to as a programming device, may be a computer, tablet, mobile device, or any other suitable device for processing information. The computing device 426 may be local to the user or may include components that are non-local to the computer including one or both of the processor 427 or memory 428 (or portions thereof). For example, the user may operate a terminal that is connected to a non-local processor or memory. In some examples, the computing device 406 may include a watch, wristband, smartphone, or the like. Such computing devices may wirelessly communicate with the other components of the electrical stimulation system, such as the CP 104, RC 103, ETM 105, or IPG 102 illustrated in FIG. 1. The computing device 426 may be used for gathering patient information, such as general activity level or present queries or tests to the patient to identify or score pain, depression, stimulation effects or side effects, cognitive ability, or the like. In some examples, the computing device 426 may prompt the patient to take a periodic test (for example, every day) for cognitive ability to monitor, for example, Alzheimer's disease. In some examples, the computing device 426 may detect, or otherwise receive as input, patient clinical responses to electrostimulation such as DBS, and determine or update stimulation parameters using a closed-loop algorithm based on the patient clinical responses, as described below with reference to FIG. 5. Examples of the patient clinical responses may include physiological signals (e.g., heart rate) or motor parameters (e.g., tremor, rigidity, bradykinesia). The computing device 426 may communicate with the axis. CP 104, RC 103, ETM 105, or IPG 102 and direct the changes to the stimulation parameters to one or more of those devices. In some examples, the computing device 426 may be a wearable device used by the patient only during programming sessions. Alternatively, the computing device 426 may be worn all the time and continually or periodically adjust the stimulation parameters. In an example, the closed-loop algorithm for determining or updating stimulation parameters may be implemented in a mobile device, such as a smartphone, that is connected to the IPG or an evaluating device (e.g., a wristband or watch). These devices may also record and send information to the clinician.

The processor 427 may include one or more processors that may be local to the user or non-local to the user or other components of the computing device 426. In an example, the processor 427 may execute instructions (e.g., stored in the memory 428) to determine a search space of electrode configurations and parameter values, and identify or update one or more stimulation settings that are selectable for use in electrostimulation therapies such as DBS. The search space may include a collection of available electrodes, possible electrode configurations, and possible values or value ranges of one or more stimulation parameters that may be applied to selected electrodes to deliver electrostimulation. The search space may be specific to a particular lead or a type of lead with respect to a specific neural target. As a result, for different leads or types of lead and/or for different neural targets, the processor 427 may determine respective different search spaces. A stimulation setting includes an electrode configuration and values for one or more stimulation parameters. The electrode configuration may include information about electrodes (ring electrodes and/or segmented electrodes) selected to be active for delivering stimulation (ON) or inactive (OFF), polarity of the selected electrodes, electrode locations (e.g., longitudinal positions of ring electrodes along the length of a non-directional lead, or longitudinal positions and angular positions of segmented electrodes on a circumference at a longitudinal position of a directional lead), stimulation modes such as monopolar pacing or bipolar pacing, etc. The stimulation parameters may include, for example, current amplitude values, current fractionalization across electrodes, stimulation frequency, stimulation pulse width, and like.

The processor 427 may identify or modify a stimulation setting from the search space through an optimization process until a search criterion is satisfied, such as until an optimal, desired, or acceptable patient clinical response is achieved. Electrostimulation programmed with a setting may be delivered to the patient, clinical effects (including therapeutic effects and/or side effects, or motor symptoms such as bradykinesia, tremor, or rigidity) may be detected, and a clinical response may be evaluated based on the detected clinical effects. When actual electrostimulation is administered, the settings may be referred to as tested settings, and the clinical responses may be referred to as tested clinical responses. In contrast, for a setting in which no electrostimulation is delivered to the patient, clinical effects may be predicted using a computational model based at least on the clinical effects detected from the tested settings, and a clinical response may be estimated using the predicted clinical effects. When no electrostimulation is delivered the settings may be referred to as predicted or estimated settings, and the clinical responses may be referred to as predicted or estimated clinical responses.

In various examples, portions of the functions of the processor 427 may be implemented as a part of a microprocessor circuit. The microprocessor circuit may be a dedicated processor such as a digital signal processor, application specific integrated circuit (ASIC), microprocessor, or other type of processor for processing information. Alternatively, the microprocessor circuit may be a processor that may receive and execute a set of instructions of performing the functions, methods, or techniques described herein.

The memory 428 may store instructions executable by the processor 427 to perform various functions including, for example, determining a reduced or restricted electrode configuration and parameter search space (also referred to as a “restricted search space”), creating or modifying one or more stimulation settings within the restricted search space, etc. The memory 428 may store the search space, the stimulation settings including the “tested” stimulation settings and the “predicted” or “estimated” stimulation settings, clinical effects (e.g., therapeutic effects and/or side effects) and clinical responses for the settings, and/or instructions for implementing a testing process for testing stimulation parameters. The memory 428 may be a computer-readable storage media that includes, for example, nonvolatile, non-transitory, removable, and non-removable media implemented in any method or technology for storage of information, such as computer readable instructions, data structures, program modules, or other data. Examples of computer-readable storage media include RAM, ROM, EEPROM, flash memory, or other memory technology, CD-ROM, digital versatile disks (“DVD”) or other optical storage, magnetic cassettes, magnetic tape, magnetic disk storage or other magnetic storage devices, or any other medium which may be used to store the desired information, and which may be accessed by a computing device.

Communication methods provide another type of computer readable media; namely communication media. Communication media typically embodies computer-readable instructions, data structures, program modules, or other data in a modulated data signal such as a carrier wave, data signal, or other transport mechanism and include any information delivery media. The terms “modulated data signal,” and “carrier-wave signal” includes a signal that has one or more of its characteristics set or changed in such a manner as to encode information, instructions, data, and the like, in the signal. By way of example, communication media includes wired media such as twisted pair, coaxial cable, fiber optics, wave guides, and other wired media and wireless media such as acoustic, RF, infrared, Bluetooth™, near field communication, and other wireless media.

The display 429 may be any suitable display or presentation device, such as a monitor, screen, display, or the like, and may include a printer. The display 429 may be a part of a user interface configured to display information about stimulation settings (e.g., electrode configurations and stimulation parameter values and value ranges) and user control elements for programming a stimulation setting into an IPG.

The input device 430 may be, for example, a keyboard, mouse, touch screen, track ball, joystick, voice recognition system, or any combination thereof, or the like. Another input device 430 may be a camera from which the clinician may observe the patient. Yet another input device 430 may a microphone where the patient or clinician may provide responses or queries.

The electrical stimulation system 400 may include, for example, any of the components illustrated in FIG. 1. The electrical stimulation system 400 may communicate with the computing device 426 through a wired or wireless connection or, alternatively or additionally, a user may provide information between the electrical stimulation system 400 and the computing device 426 using a computer-readable medium or by some other mechanism.

FIG. 5 illustrates, by way of example and not limitation, a stimulation parameter control system and a part of the environment in which it may operate. The stimulation parameter control system 531, which may be implemented as a part of the processor 427 in FIG. 4, may include a feedback control logic 532, a DBS controller 533, and a search space identifier 534. DBS is used as an example. It is noted that the system may be implemented for other stimulation therapies such as, but not limited to, SCS or PNS. The feedback control logic 532 may be implemented in, for example, the CP 104 or the RC 103 in FIG. 1. The feedback control logic 532 may determine or modify one or more stimulation settings 535 for a stimulation lead at a target stimulation region, such as a region in a brain hemisphere. A stimulation setting may include an electrode configuration and values for one or more stimulation parameters (P₁, P₂, . . . , Pₘ). The electrode configuration includes information about electrodes (ring electrodes and/or segmented electrodes) selected to be active for delivering stimulation (ON) or inactive (OFF), polarity of the selected electrodes, electrode locations (also referred to as contact locations, which may include longitudinal positions of ring electrodes along the length of a lead, or angular positions of segmented electrodes about a circumference of a cross-section of the lead at a longitudinal position), and stimulation modes (e.g., monopolar pacing or bipolar pacing), etc. The stimulation parameters may include, for example, current amplitude values, current fractionalization across electrodes, stimulation frequency, stimulation pulse width, etc. In some examples, the feedback control logic 532 may modify the stimulation setting 535 such as by changing a stimulation parameter value, or modifying an electrode configuration.

The stimulation setting 535 may be provided to the DBS controller 533 to configure the IPG or ETM to deliver DBS therapy to the patient 536 in accordance with the stimulation setting or the modified stimulation setting. The stimulation may produce certain therapeutic effects and/or side effects on the patient 536. Such therapeutic effectiveness and side effects, also referred to as clinical responses or clinical metrics, may be provided to the feedback control logic 532. In an example, the clinical responses may be based on patient or clinician observations. For example, motor symptoms such as bradykinesia (slowness of movement), rigidity, tremor, among other symptoms or side effects, may be scored by the patient or by the clinician upon overserving or questioning the patient. In some examples, the clinical responses may be objective in nature, such as measurements automatically or semi-automatically taken by a sensor 537. In an example, the sensor 537 may be included in a wearable device associated with patient 536, such as a smart watch. For example, a Parkinson's patient may be fitted with a wearable sensor that measures tremors, such as by measuring the frequency and amplitude of such tremors.

The clinical responses, either reported by the patient or measured by a sensor, may be converted to clinical response values 538, also referred to as clinical response scores. In an example, the clinical response values 538 may be computed based on the intensity, frequency, or duration of one or more of tremor, rigidity, or bradykinesia responses. Based upon the received clinical response values 538, the feedback control logic 532 may adjust electrode configurations or values of one or more stimulation parameters 535. The feedback control logic 532 may send the adjusted (new or revised) stimulation setting 535, such as the electrode configuration or the adjusted stimulation parameter values, to further configure the DBS controller 533 to change the stimulation parameters of the leads implanted in patient 506 to the adjusted values.

The feedback-control loop as illustrated in FIG. 5 may continue until an optimal, desired, or acceptable outcome is reached, such as maximizing therapeutic effectiveness while minimizing unwanted side effects, or until a specific stop condition is reached such as number of iterations, time spent in programming session, or the like. An outcome may be considered optimal, desired, or acceptable if it meets certain threshold values or tests (e.g., improved clinical response for the patient, faster programming of the device, increased battery life, and/or control multiple independent current sources and directional lead). Such an iterative process of looking for a stimulation setting (e.g., an electrode configuration and stimulation parameter values for the electrode) is referred to as a stimulation setting optimization process. The outcome being reached may be referred to as an optimization criterion, and the resultant stimulation setting may be referred to as an optimal base stimulation setting (BSS). By way of example and not limitation, the optimization criterion may include possible optimal clinical outcome within the parameters chosen; time spent, iterations taken, or power usage to explore the search space until a desired clinical outcome is reached (assuming multiple outcomes with the same or comparable clinical response); among others.

In an example, the optimization criterion includes the clinical response values 538 exceeding a threshold value or falling into a specified value range, indicating a satisfactory therapeutic outcome has reached. Depending on how the clinical response values are computed, one or more optimal base stimulation settings may be determined. For example, the clinical response values may be computed using a single response effect (e.g., one of bradykinesia, tremor, or rigidity). Accordingly, three optimal base stimulation settings may be generated: a first optimal base stimulation setting (BSS₁) corresponding to a bradykinesia score exceeding a threshold, a second optimal base stimulation setting (BSS₂) corresponding to a tremor score exceeding a threshold, and a third optimal base stimulation setting (BSS₃) corresponding to a rigidity score exceeding a threshold. In another example, the clinical response values may be a composite score computed as a weighted combination of multiple clinical effects, such as a %*bradykinesia+b %*tremor+c %*rigidity. Accordingly, a fourth optimal base stimulation setting (BSS₄) may be generated, corresponding to the composite clinical response score exceeding a threshold. In some examples, the stimulation setting optimization may be performed in an in-clinic programming session such during implantation or revision of a DBS system or device follow-up.

The optimal base stimulation settings (e.g., BSS₁ through BSS₄), may be stored in the memory 528. In an example, a stimulation setting, along with the corresponding unique clinical response indicator (e.g., weighted combination of clinical effects with unique weight factors) form a stimulation program 539, which may also be stored in the memory 404. Each stimulation programmed may be associated with, or tagged by, one or more unique clinical response indicators. In some examples, the clinical response values 538 may be weighted according to the time at which the test took place.

In various examples, the stimulation parameter control system 531 may be executed on its own and is not connected to a controller. In such instances it may be used to merely determine and suggest programming parameters, visualize a parameter space, test potential parameters, etc.

The process of searching for a stimulation setting (e.g., an electrode configuration and/or stimulation parameter values) typically involves significant computation and time, especially when electrode configuration involves segmented electrodes in a directional lead. If testing all possible settings in the entire parameter space (including electrode configurations and combinations of stimulation parameter values) is done as comprehensively as possible, stimulation would need to be provided to the patient for each possible setting, which may end up with a burdensome and time-consuming programming session. Because practically a programming session may only last a few hours, only a fraction of possible electrode configuration and stimulation parameter combinations may reasonably be tested and evaluated. To reduce the time taken and to improve the efficiency of stimulation setting optimization process, a reduced or restricted electrode configuration and parameter search space may be used. By applying limitations or constraints to the electrode configurations and parameter values, the restricted search space may include a subset of electrodes (e.g., a subset of ring electrodes and/or a subset of segmented electrodes on a lead) that are selected as active electrodes for delivering stimulation, and values or value ranges for one or more stimulation parameters (e.g., a range of current amplitude ranges for an active electrode). Stimulation setting optimization, when performed within such a search space, may be more efficient and cost-effective than searching through the entire parameter space for one or more optimal base stimulation settings such as BSS₁-BSS₄as discussed above.

The search space identifier 534 may automatically determine a search space 540 for a stimulation lead at a neural target, such as a region in a brain hemisphere, by imposing certain limitations or constraints on the electrode configurations and/or parameter values or value ranges. In an example, the search space 540 may be determined based on spatial information of the lead, such as lead positions with respect to neural targets, which may be obtained from imaging data of the lead and patient anatomy. Additionally, or alternatively, the search space 540 may be determined based on physiological information such as physiological signals sensed by the electrodes at their respective tissue contact locations. The physiological information may include patient clinical responses to stimulation. In some examples, prior knowledge about patient medical condition, health status, DBS treatment history may be used to determine the search space 540. In an example, the search space identifier 534 may exclude those electrodes on the lead that are out of a region of interest, such that the search space includes only those electrodes within the target of interest. One or more stimulation parameters may be restricted to take certain values or within value ranges. For example, the restricted search space may include certain electrode positions and value ranges for stimulation current amplitude, frequency, or pulse width. The feedback control logic 532 may determine one or more optimal base stimulation settings (e.g., BSS₁-BSS₄) by searching through the identified search space 540. The identified search space 540 may be stored in the memory 528.

The feedback control logic 532 may include a machine learning engine 541 that may facilitate the stimulation parameter control system 531 (or a user of the system) to explore the search space in order to choose values for programming the DBS controller 533. The machine learning engine 541 may employ supervised or unsupervised learning algorithms to train a prediction model, and use the trained prediction model to predict patient clinical responses to an untested stimulation setting (e.g., untested stimulation parameter values or untested electrode configurations), or to estimate or predict stimulation parameters values or electrode configurations that, when provided to the DBS controller 533 to deliver stimulation accordingly to the patient 536, would produce desired or improved clinical responses. Examples of the learning algorithms include, for example, Naive Bayes classifiers, support vector machines (SVMs), ensemble classifiers, neural networks, Kalman filters, regression analyzers, etc. The machine learning engine 541 may build and train a prediction model using training data, such as stimulation parameter values and corresponding patient clinical responses. The training data may be acquired from a training session such as performed in a clinic. Additionally, or alternatively, the training data may be obtained from historical data acquired by the stimulation parameter control system 531. With its learning and prediction capability, the machine learning engine 541 may aid a user (e.g., a clinician) in exploring the stimulation parameter space more effectively and more efficiently to produce results that are optimal, desired, or acceptable.

In some examples, the machine learning engine 541 may use imaging data to inform the choice of the next set of values, which may be used when the algorithm finds itself in a region of parameter space for which the clinical responses are not substantially affected by the changes in the stimulation parameters, and the choice of next step is not apparent from the patient response alone. Imaging data that provides information about the location of the lead in the patient's brain along with priors informing the algorithm of which directions may be better choices for the next step could lead to faster convergence.

In some examples, the machine learning engine 541 may determine expected outcomes for parameter values that have not yet been tested based upon what the machine learning engine 541 has “learned” thus far and provide a recommendation for a next set of values to test. Here, testing refers to the iterative testing required to find an optimal stimulation setting for configuring the DBS controller 533. The recommendation for a next set of values to test is based upon which of the determined expected outcomes meet a set of designated (determined, selected, preselected, etc.) criteria (e.g., rules, heuristics, factors, and the like). For example, rules considered may include such factors as: the next set of values may not be one of the last 10 settings tested or may not be too close to previously tested setting. Accordingly, the feedback control logic 532 with its machine learning engine 541 is used to systematically explore the stimulation parameter space based upon what it has learned thus far and (optionally) different rules and/or heuristics that contribute to achieving optimal outcomes more efficiently.

The process for determining expected outcomes for parameter values that have not yet been tested may involve use of other data for machine learning. For example, data from other programming sessions for the same patient as well as from other patients may be used to train the machine learning engine 541. In some examples, no prior data may be used. In this case, the machine learning engine 541 may use data learned from this patient only in one particular setting. In other examples, data from the same patient but from previous sessions may be used. In some examples all patient data from all sessions may be used. In some examples all patient data utilizing lead location information (knowledge of lead location in space relative to anatomy) may be used. Different other combinations are also possible.

In order to use this data for machine learning purposes, the data may first be cleansed, optionally transformed, and then modeled. In some examples, new variables are derived, such as for use with directional leads, including central point of stimulation, maximum radius, spread of stimulation field, or the like. Data cleansing and transformation techniques such as missing data imputation and dimension reduction may be employed to prepare the data for modeling.

The machine learning engine 541 may determine how best a predicted outcome meets the optimal outcome metrics. Various optimization techniques may be used, examples of which may include but are not limited to: optimization algorithms and estimation procedures used to fit the model to the data (e.g., gradient descent, Kalman filter, Markov chain, Monte Carlo, and the like); optimization algorithms reformulated for search (e.g., simulated annealing); spatial interpolation (e.g., kriging, inverse distance weighting, natural neighbor, etc.); supplementary methods that aid the optimization process (e.g., variable selections, regularization, cross validation, etc.); other search algorithms (e.g., golden-section search, binary search, etc.). Using any of these techniques, the machine learning engine 541 may decide whether a particular predicted outcome for a set of stimulation parameter values is the fastest sufficing outcome, the best possible clinical outcome, or the optimal outcome with least battery usage, for example.

The feedback control logic 532 may be used to search and configure different types of stimulation parameters of the various leads potentially causing different clinical effects upon the patient 536. Examples of the stimulation parameters may include electrode configurations (electrode selection, polarities, monopolar or bipolar modes of stimulation), current fractionalization, current amplitude, pulse width, frequency, among others. Given these possible stimulation parameters, the stimulation parameter control system 531 may move about the parameter space in different orders, by different increments, and limited to specific ranges. In some examples, the stimulation parameter control system 531 may allow the user to provide search range limitations to one or more of the stimulation parameters to limit the range for that stimulation parameter over which the system will search for parameters. For example, the user may restrict which electrodes may be used for stimulation or may restrict the amplitude or pulse width to a certain range or with a selected maximum or minimum. As one illustration, based on the site of implantation, the user may be aware that the distal-most and proximal-most electrodes are unlikely to produce suitable stimulation and the user limits the range of electrodes to exclude these two electrodes.

For a lead with segmented electrodes, the number of possibilities for parameter selection may be very large when combinations of electrodes and different amplitudes on each electrode are possible. In some examples using a lead with segmented electrodes, the selection of electrodes used for stimulation may be limited to fully directional selections (i.e., selection of only a single segmented electrode) and fully concentric selections (i.e., all electrodes in a single set of segmented electrodes are active with the same amplitude). In other examples, the initial movement through parameter space may be limited to fully directional and fully concentric selections. After a set of stimulation parameters is identified using these limits, variation in the selection of electrodes may be opened up to other possibilities near the selection in the identified set of stimulation parameters to further optimize the stimulation parameters.

In some examples, the number of stimulation parameters that are varied and the range of those variations may be limited. For example, some stimulation parameters (e.g., electrode selection, amplitude, and pulse width) may have larger effects when varied than other stimulation parameters (e.g., pulse shape or pulse duration). The movement through stimulation parameter space may be limited to those stimulation parameters which exhibit larger effects. In some examples, as the stimulation parameter control system 531 proceeds through testing of sets of stimulation parameters, the system may observe which stimulation parameters provide larger effects when varied and focus on exploring variation in those stimulation parameters.

In some examples, the stimulation parameter control system 531 may include a user interface for visualizing exploration of the stimulation parameter space as the system determines new and better parameter values to test until a solution is determined that fits within certain designated thresholds or a stop condition is reached. In some examples of the stimulation parameter control system 531, the user interface is part of the feedback control logic. In other examples, the user interface may be part of another computing system that is part of the stimulation parameter control system 531 or may be remote and communicatively connected to the stimulation parameter control system 531. The user interface may present to a user (such as a clinician, physician, programmer, etc.) a visualization of the predicted expected outcomes for (some of) the stimulation parameter values not yet tested and a recommendation for the next set of stimulation parameter values to test.

In some examples where a deep brain stimulator is configured via the DBS controller 533 with at least one set of stimulation parameter values forwarded by the feedback control logic 532, the clinician may monitor the patient throughout the process and record clinical observables in addition to the patient 536 being able to report side effects. When a side effect is observed, the various search algorithms may take that fact into account when selecting/suggesting a next set of values to test. In some examples, for example, those that select contacts via monopolar review, other parameters may be changed until they cause a side effect, which case is noted as a boundary. For example, in monopolar review where amplitude is another stimulation parameter being varied, the amplitude may be increased progressively until a side-effect is observed.

In some examples, more than one clinical metric (e.g., tremor, rigidity, bradykinesia, etc.) may be important observables. Different examples of the stimulation parameter control system 531 may handle these metrics differently. For example, some examples might identify an ideal location for each metric and choose one ideal location between them, set in the patient's remote controller so the patient may choose as needed, or chose a best combined outcome. As another example, some examples may search multiple outcomes at the same time and use the best combined score as the best outcome or find a best location for each metric individually. As yet another example, some examples may use a sequential process for selecting stimulation parameter values for multiple outcomes. For example, a system may search parameter space for a first outcome (e.g., bradykinesia) and, upon finding a suitable end condition, then search parameter space for a second outcome (e.g., rigidity). While searching parameter space for the first outcome, clinical response values for both the first and second outcomes may be obtained. Thus, when the system switches to the second outcome there are already a number of clinical response values for that outcome which will likely reduce the length of the search.

In some examples, two stimulation leads may be implanted to produce stimulation effects on two sides of the body (e.g., the right and left sides of the body). The same procedure described herein may be used to either jointly determine the stimulation parameters for the two leads by exploring the joint parameter space or individually determine stimulation parameters for the two leads by exploring the parameter space for each lead individually. In some examples, the user may determine for each side of the body which clinical response is dominant or most responsive. This may be done, for example, by having the patient perform a single task which captures multiple responses (e.g., connecting dots on the screen to monitor tremor and bradykinesia of the movement) or a small series of tasks. This enables the system to determine which clinical response to use to identify the stimulation parameters for that side of the body.

As noted, the feedback may be provided directly by the patient 536, entered by an observer such as a clinician (not shown), or may be provided by means of a sensor 537 associated with and in physical, auditory, or visual contact with the patient 536. Examples may include, but are not limited to, accelerometers, microphones, and cameras. In an example, the sensor 537 may be included in a wearable device associated with patient 536, such as a smart watch. In an example where the feedback may be monitored automatically or semi-automatically, such as with use of sensor 537, it may not be necessary for a clinician or other observer to be present to operate the stimulation parameter control system 531. Accordingly, in such examples a user interface may not be present in system 531.

In some examples, the stimulation parameter control system 531 may determine one or more optimal base stimulation settings using predicted clinical responses for untested stimulation parameter values or untested electrode configurations without actually delivering stimulation. Such base stimulation settings are referred to as estimated or predicted base stimulation settings, to distinguish from the tested base stimulation settings (e.g., BSS₁-BSS₄) that are based on the tested clinical response (either reported by the patient or measured by a sensor) to actually delivered stimulation. For examples, based on the “tested” base stimulation settings BSS₁-BSS₄, the stimulation parameter control system 531 may estimate an optimal base stimulation setting associated with a composite clinical response defined as x %*bradykinesia+y %*tremor+z %*rigidity, or simply denoted by the weight factors (x %, y %, z %). By way of example and not limitation, the stimulation parameter control system 531 may generate a fifth optimal base stimulation setting (BSS₅) corresponding to a composite clinical response using bradykinesia and tremor only, each weighted 50%; a sixth optimal base stimulation setting (BSS₆) corresponding to a composite clinical response using tremor and rigidity only, each weighted 50%; a seventh optimal base stimulation setting (BSS₇) corresponding to a composite clinical response using bradykinesia and rigidity only, each weighted 50%; or an eighth optimal base stimulation setting (BSS₈) corresponding to a composite clinical response using bradykinesia, tremor, and rigidity weighted 40%, 40%, and 20%, respectively. Similar to the tested base stimulation settings BSS₁-BSS₄, the estimated base stimulation settings BSS₅-BSS₈, associated with their respective clinical response indicators (e.g., weight factors for clinical effects), may be stored in the memory 528 as respective stimulation programs 539. In an example, the stimulation programs 539 may be stored in a lookup table, where each tested or estimated base stimulation setting (e.g., BSS₁ through BSS₈) may be tagged by respective clinical response indicators or weight factors for clinical effects. In an example, the memory 528 may be a part of memory circuitry internal to the IPG. The RC or the CP may request access to the memory 528 to retrieve therefrom one or more stored stimulation programs 539 or the search space 540.

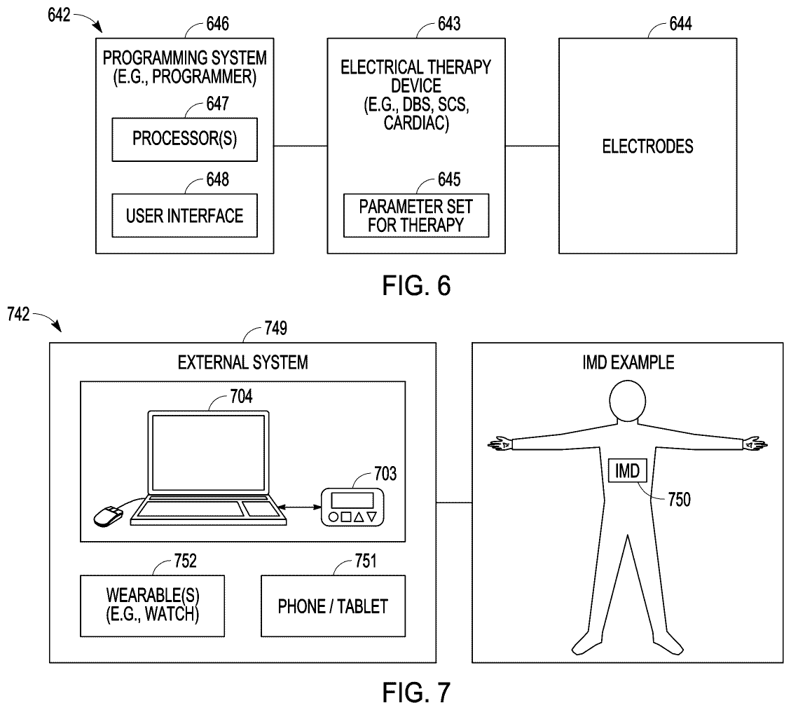

FIG. 6 illustrates, by way of example, an example of an electrical therapy-delivery system. The illustrated system 642 includes an electrical therapy device 643 configured to deliver an electrical therapy to electrodes 644 to treat a condition in accordance with a programmed parameter set 645 for the therapy. The system 642 may include a processing system 646 that may include one or more processors 647 and a user interface 648, which may be used to program and/or evaluate the parameter set(s) used to deliver the therapy. The illustrated system 642 may be a DBS system for treating a movement disorder, such as has been illustrated and discussed with respect to FIGS. 1-5, and/or a system for monitoring the movement disorder.

In some embodiments, the illustrated system 642 may include an SCS system to treat pain and/or a system for monitoring pain. By way of example, a therapeutic goal for conventional SCS programming may be to maximize stimulation (i.e., recruitment) of the dorsal column (DC) fibers that run in the white matter along the longitudinal axis of the spinal cord and minimal stimulation of other fibers that run perpendicular to the longitudinal axis of the spinal cord (e.g., dorsal root fibers).

FIG. 7 illustrates, by way of example and not limitation, the electrical therapy-delivery system of FIG. 6 implemented using an implantable medical device (IMD). The illustrated system 742 includes an external system 749 that may include at least one programming device. The illustrated external system 749 may include a clinician programmer 704, similar to CP 104 in FIG. 1, configured for use by a clinician to communicate with and program the neuromodulator, and a remote control device 703, similar to RC 103 in FIG. 1, configured for use by the patient to communicate with and program the neuromodulator. For example, the remote control device 703 may allow the patient to turn a therapy on and off and/or may allow the patient to adjust patient-programmable parameter(s) of the plurality of stimulation parameters. FIG. 7 illustrates an IMD 750, although the monitor and/or therapy device may be an external device such as a wearable device. The external system 749 may include a network of computers, including computer(s) remotely located from the IMD 750 that are capable of communicating via one or more communication networks with the programmer 704 and/or the remote control device 703. The remotely located computer(s) and the IMD 750 may be configured to communicate with each other via another external device such as the programmer 704 or the remote control device 703. The remote control device 703 and/or the programmer 704 may allow a user (e.g., patient and/or clinician or rep) to answer questions as part of a data collection process. The external system 749 may include personal devices such as a phone or tablet 751, wearables such as a watch 752, sensors or therapy-applying devices. The watch may include sensor(s), such as sensor(s) for detecting activity, motion and/or posture. Other wearable sensor(s) may be configured for use to detect activity, motion and/or posture of the patient. The external system 749 may include, but is not limited to, a phone and/or a tablet. The phone and/or tablet may include camera(s), microphone(s), accelerometer(s) or other sensors that can be used to provide feedback. The system may include physical sensors. In nonlimiting examples, the physical sensor may be configured for use in detecting or determining rigidity, stiffness, muscle tension, or movement. The physical sensors may include internal physical sensors or external physical sensors.

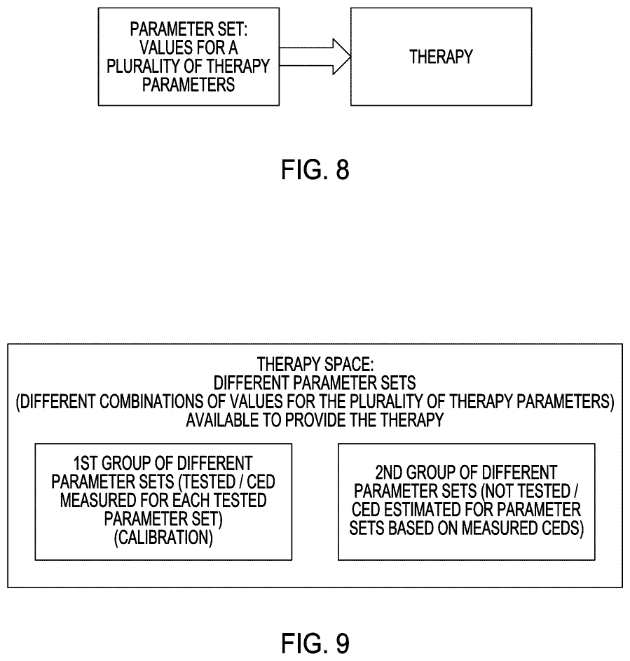

FIG. 8 illustrates a therapy being delivered according to a parameter set. The parameter set may be programmed into the device to deliver the specific therapy using specific values for a plurality of therapy parameters. For example, the therapy parameters that control the therapy may include pulse amplitude, pulse frequency, pulse width, and electrode configuration (e.g., selected electrodes, polarity and fractionalization). The parameter set includes specific values for the therapy parameters.

FIG. 9 illustrates a therapy space, which includes different parameter sets potentially available for delivering the therapy. The different parameter sets have unique combinations of values for the therapy parameters. The therapy space may be burdensomely large as there may be many unique combinations of values for therapy parameters (e.g., many unique parameter sets). Some parameter sets within the therapy space may be tested and the corresponding clinical effect data (CED) may be measured or otherwise acquired for the tested parameter sets. These tested parameter sets are illustrated as a first group of different parameter sets within the therapy space. Other parameter sets may not be tested (e.g., second group of different parameter sets). The CED for these parameter sets may be estimated based on measured CEDs for the patient or a patient population.

For example, CED may be directly measured to provide calibration settings. The therapy sessions may be delivered using different therapy settings, and the CED may be recorded for each session. For a neurostimulator such as DBS, SCS, PNS or TENS, for example, the therapy may involve delivering electrical waveforms, which may be a pulsed waveform. Programmable settings for the pulse waveform may include a pulse amplitude, a pulse width, a pulse frequency, a pulse train duration, a pulse-to-pulse duty cycle, a pulse train to pulse train duty cycle (stimulation ON/OFF), and a stimulation schedule (e.g., programmable start and/or stop times, such as but not necessarily a calendar-based schedule). The programmable settings may further include controlling which of a plurality of electrodes are active and which are off, the polarity of each active electrode (which active electrode(s) are anode(s) and which are cathode(s), and the contributions (e.g., electrode fractionalization) of total energy delivered to individual one(s) of the anode(s) and individual one(s) of the cathode(s). Thus, by way of example and not limitation, one electrode may be programmed to provide all (100%) of the anodic energy, and four electrodes may be programmed to provide fractions (e.g., 25%, 25%, 25%, 25%; or 10%, 20%, 30% and 40%) of the total cathodic energy. Controlling the individual contributions by individual electrodes adjusts the location and shape of the stimulation field, to modulate different combinations of neural elements. The settings may be spread throughout the stimulation space for use in identifying clinical responses from session to session, including both the previously-measured clinical effects for tested stimulation settings and predicted or estimated clinical effects for stimulation settings that were not previously tested.

Given the large number of available parameters sets, it is not practical to test all possible parameter sets to map the corresponding stimulation effects for the parameter sets. However, based on what has been tested, stimulation effect predictions (e.g., estimated responses) may be made for parameter sets that were not tested. By way of example and not limitation, these predictions may be used to determine a good parameter set for testing or for therapy.

The responses or stimulation effects for tested parameter sets may include patient responses associated with patient outcomes which may be referred to as clinical effects data. Patient outcomes may include perception, therapeutic effects, and side effects. The responses or stimulation effects for tested parameter sets may also include sensed data. For a system that is capable of sensing, not only are only some of the available stimulation sets tested, but sensing may have been performed for only some of the tested stimulation sets. The sensing data for these parameter sets may be used to extrapolate or interpolate sensed data predictions for parameter sets that are not associated with sensed data. Some of the tested stimulation sets may be associated with patient outcomes such as patient symptoms (e.g., clinical effects data). Some of the tested stimulation sets may be associated with both sensed data and patient outcomes, some tested stimulation sets may be associated with only patient outcomes without patient data, and some tested stimulation sets may be associated with only sensed data without patient outcomes. A stimulation effects map may include a combination of sensing data and patient outcomes, both tested and predicted, which may provide more information about stimulation parameter sets that should be tested or that may even be used in a program to treat the patient.

There may be many parameters in a set that may be tested. In order to assist with visualization, the different parameter sets may correspond to different stimulation locations, and some of these stimulation locations may be tested. These tested locations may be used to make predictions for other locations.

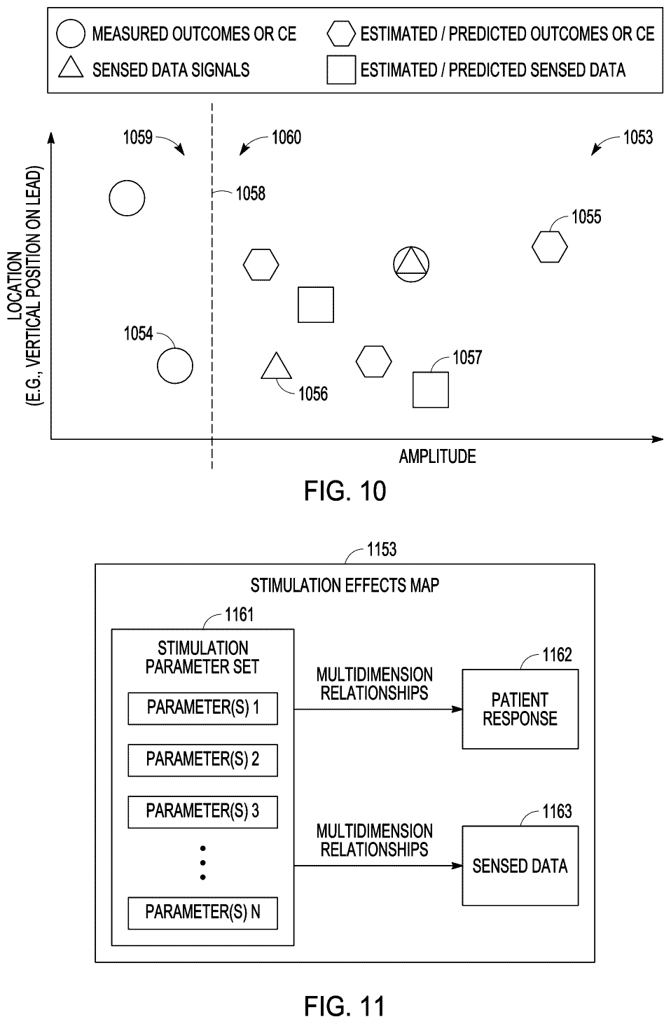

FIG. 10 illustrates, by way of example and not limitation, a two-dimensional plot of location and amplitude for measured and estimated outcomes (e.g., clinical effects) and sensed and estimated data. This map 1053 combines both the amplitude of the neurostimulation on the X-axis and the position of the neurostimulation on the lead on the Y-axis. It is noted that the map may include information for additional stimulation parameter such as, but not limited to, pulse width and pulse rate. Therefore, multidimensional maps may be implemented.

In the illustrated map 1053, the circles 1054 represent measured, observed or experienced patient outcomes (e.g., clinical effects) for at least some tested parameter sets and the hexagons 1055 represent estimated patient outcomes for at least some untested parameter sets. The patient outcomes may be entered in response to queries on a user device. The patient outcomes may be a side effect, a therapeutic effect, or other outcome. Thus, for example, the patient outcomes may be improvements in a symptom and/or a side effect. By way of example and not limitation, the circles may represent motor testing results for various DBS therapy parameter sets. The triangles 1056 may represent the sensed data for some tested stimulation parameter sets. and the squares 1057 represent estimated sensed data for at least some of the untested parameter sets.

The dotted line 1058 may represent a threshold for the stimulation effect that is being mapped, where points on one side 1059 represent parameter combinations that do not produce the stimulation effect on points on the other side 1060 represent parameter combinations that do produce the stimulation effect. For a simpler two-dimensional map, the dotted line may simply be illustrated as a vertical line. However, the line may have different slopes and need not be a straight line but rather may have various inflections. Multi-dimensional maps may have a multidimensional function separating parameter sets that do not provide the stimulation effect from parameter sets that do provide the stimulation effect.

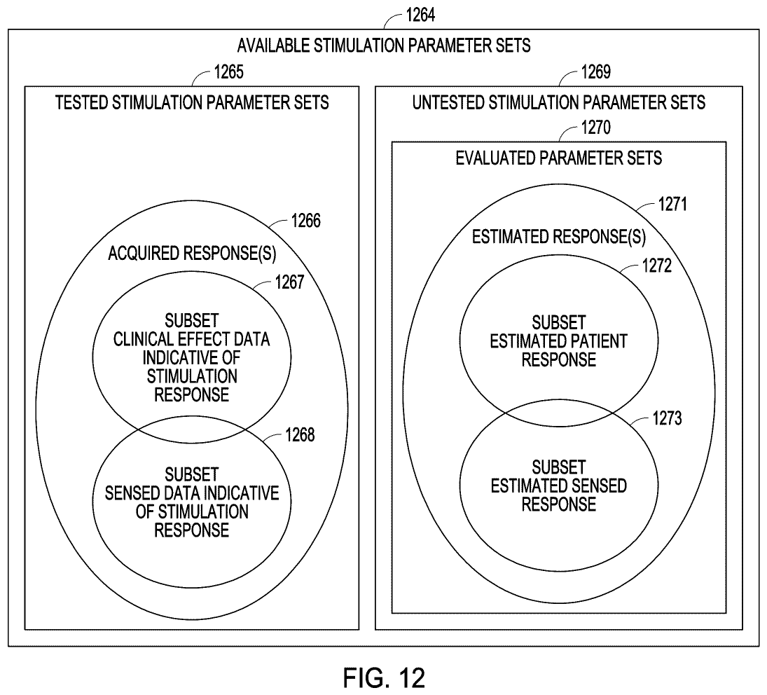

FIG. 11 illustrates, by way of example and not limitation, a stimulation effects map 1153 having multidimension relationships between a stimulation parameter set 1161 (illustrated as including N parameter(s)) and each of a patient response 1162 and sensed data 1163. Stimulation effects (e.g., a patient response and/or sensed data) may be associated with different stimulation parameter sets with three or more stimulation parameter(s). The parameter set includes “parameter(s)” that may be associated with the patient response and/or sensed data, as the association may be for one parameter or for a composite parameter such as “charge” where charge corresponds to a combination of pulse amplitude and pulse width.

FIG. 12 illustrates, by way of example and not limitation, available stimulation parameter sets. The available parameter sets 1264 may include an electrode configuration and a stimulation waveform configuration. The electrode configuration may at least partially control the location of the stimulation field and may include parameters such as a selection of activated electrodes and the polarity of active electrodes fractionalization across the active electrode. The stimulation waveform configuration may include waveform parameters such as, but not limited to, amplitude, frequency and pulse width. Some of the available stimulation parameter sets 1264 are tested (see tested stimulation parameter sets 1265) by delivering neurostimulation using corresponding parameter values, and acquiring responses to the neurostimulation 1266. The acquired responses 1266 for a tested parameter set 1265 may include a subset of clinical effect data 1267 indicative of a stimulation response (e.g., clinical effect) such as at least one patient symptom. The acquired responses 1266 for a tested parameter set 1265 may include a subset of sensed data indicative of a stimulation response 1268. By way of example and not limitation, sensed data may correspond to patient movement sensed using an accelerometer and/or camera. The acquired response subsets 1267 and 1268 may be exclusive or nonexclusive. For example, a single evaluated parameter set may only be associated with acquired clinical effect data 1267, only associated with acquired sensed data 1268, or may be associated with both acquired clinical effect data 1267 and acquired sensed data 1268. Some of the available stimulation parameter sets 1264 are not tested (see untested stimulation parameter sets 1269), and some of these untested stimulation parameter sets 1269 are evaluated 1270 by predicting or estimating the response(s) 1271. For example, the estimations may be based on extrapolating or interpolating the estimated response using acquired responses and may also use other estimated response(s). The estimated response(s) 1271 for an evaluated parameter set 1270 may include a subset of estimated patient response(s) 1272 and a subset of estimated sensed response(s) 1273. The estimated response subsets 1272 and 1273 may be exclusive or nonexclusive. For example, a single evaluated parameter set 1270 may only be associated with estimated patient response(s) 1272, only associated with estimated sensed response(s) 1273, or associated with both estimated patient response(s) 1272 and estimated sensed response(s) 1273. The number of acquired responses may be sufficient to interpolate or extrapolate to provide the estimated response(s).

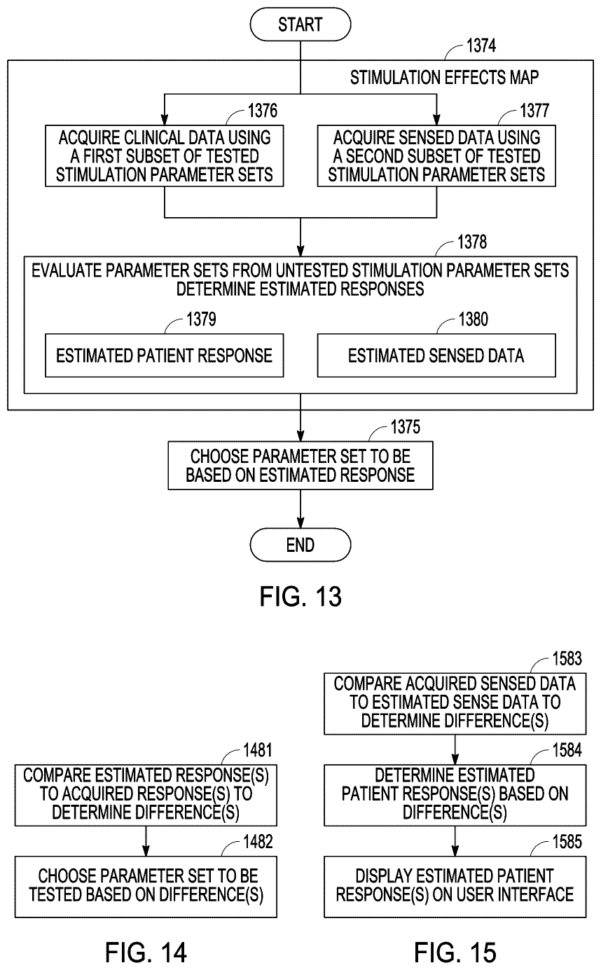

FIG. 13 illustrates, by way of example and not limitation, a method for providing a stimulation effects map 1374 which may be used to choose a parameter set to be tested or used for therapy 1375. The map may be provided by testing stimulation parameter sets from a plurality of available stimulation parameter sets by controlling the neurostimulator for each of the tested stimulation parameter sets to deliver the electrical energy using the corresponding stimulation parameter set. The map 1374 may be provided by acquiring clinical effect data indicative of a patient response to the electrical energy delivered to the tissue using at least a first subset of the tested stimulation parameter sets 1376, acquiring sensed data indicative of a sensed response to the electrical energy delivered to the tissue using at least a second subset of the tested stimulation parameter sets 1377, and evaluating parameter sets from the plurality of untested stimulation parameter sets to provide evaluated parameter sets 1378. Each of the evaluated parameter sets 1378 may be provided by estimating at least one of the patient response 1379 or the sensed response 1380 to the electrical energy by interpolating, extrapolating or otherwise using the acquired clinical effect data 1376 and the acquired sensed data 1377. The stimulation effects map 1374 may include the acquired clinical effect data 1376, the acquired sensed data 1377, and the estimated responses for the evaluated parameter sets 1378.

In a nonlimiting example, the stimulation effects map 1375 may interpolate or extrapolate therapeutic thresholds or side effect threshold to track or predict therapeutic parameters or side effect parameters. By way of example and not limitation, the features of a sensed signal can be measured at a multitude of points (1377) and a predictive algorithm can be used to estimate these features at untested locations (e.g., 1380). The estimation may include interpolation and extrapolation via line and surface fitting. The estimation may be at least partially based on prior knowledge about expected topology of the stimulation response(s) within the search, parameter or other space, where the expected topology may include slopes and orientations, peaks, valleys, cliffs, or plateaus within the search space. In DBS, for example, a cliff in the therapy may be a certain amplitude of stimulation which causes a side effect and all amplitudes (or charge delivery) above the amplitude will also cause the side effect. A cliff may be identified using testing or determined anatomy using prior data from the current patient and/or a patient population.

Medical imaging may be used to determine anatomy. Anatomy may help inform that prior data for anything previously tested. For example, if a very low amplitude side effect is determined for one region, it may be predicted that lower amplitude side effects may occur at other regions of the lead as well. The lead may be orientated in a direction which could affect the dotted line in FIG. 10 or function demarcating the region where the stimulation effect occurs or where the region does not occur.

FIG. 14 illustrates, by way of example and not limitation, a method for choosing a parameter set to be tested based on a difference between estimated response(s) and acquired response(s). The illustrated method includes comparing estimated response(s) to acquired response(s) to determine difference(s) 1481 and choosing a parameter set(s) to be tested based on the difference(s) 1482. Features of a sensed signal may be used to determine or rate the degree of difference between two different stimulation parameter sets. The degree of difference from the sensed signal and estimated sensed signals may be used as part of a method to select a next setting to test

FIG. 15 illustrates, by way of example and not limitation, a method for displaying an estimated patient response determined based on a difference between sensed and estimated sense data. The method may include comparing sensed data to estimated sensed data to determine a difference as illustrated at 1583. The determined difference may be used to determine an estimated patient response as illustrated at 1584, and the estimated patient response may be displayed on a user interface as illustrated at 1885. The degree of difference from sensed signals and estimated sensed signals can be displayed to a user on a UI interface to indicate the degree of expected change (therapy, side effect) between (two) settings. Displaying the estimated response may help the user understand what change might constitute as minimal, moderate, or significant compared to a current setting being used, which may allow the user to be more informed about how they can anticipate perceiving the treatment change and may help prevent a user from unknowingly making an extreme change to their therapy unintentionally

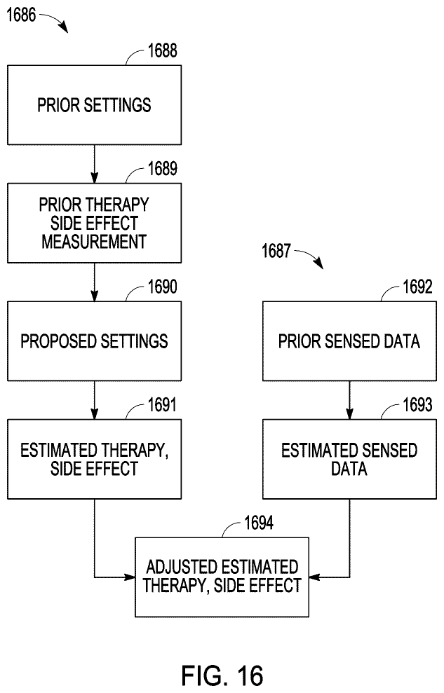

FIG. 16 illustrates, by way of example and not limitation, a method for updating estimated responses. There may be two different pathways for previously tested stimulation settings. The first pathway 1686 may involve programming algorithms based on observed or experienced patient responses, and the second pathway 1687 may involve sensed data indicative of patient responses. The first pathway 1686 may have prior stimulation settings 1688, and the therapy may be delivered at 1689 using the prior settings and symptom relief and/or a side effect measurement taken. An estimated or proposed setting may be determined using the symptom relief and/or a side effect measurement at 1690, and the estimated patient response (e.g., therapy or side effect) may be provided at 1691. The second pathway 1687 may have prior sensed data 1692 and estimated sensed data may be provided at 1693. The estimated response(s) may be based on a model for predicting a response from acquired responses. Predictions or estimations for parameter set(s) may be compared to actual sensed data and/or patient outcomes for the same parameter set(s) or adjacent parameter sets. This comparison may be used to gauge whether the prediction was good and may be used to improve the prediction model 1694 used to adjust the estimations. Artificial Intelligence (AI) or Machine Learning (ML) analysis across multiple recordings for one or many patients can be used to generate predictions. For example, the AI/ML analysis may involve determining and revising a function that demarcates parameter sets that provide a stimulation effect and parameters that do not provide the stimulation effect. Information inputted into the AI/ML analysis may include sensed data and/or observed or experienced patient responses for one or more various stimulation effects, other estimates, and anatomy information and location. Aggregate data may be collected from other patients who have been treated using neurostimulation for similar indications. For example, a patient application can be installed on a patient's smartphone to present a questionnaire and receive answers and/or to receive signals sensed by one or more sensors worn by the patient. A remote controller can be provided to the patient for adjusting delivery of neurostimulation from an implantable stimulator placed in the patient. The remote controller can receive signals sensed by the implantable stimulator and record adjustments to the settings of the implantable neurostimulators made by the patient. The aggregate data can include data indirectly collected from the patients. For example, an external device (e.g., programmer) communicating with an implantable stimulator placed in a patient can receive signals sensed by the implantable stimulator, receive information tracking operations of the implantable stimulator, record settings of the implantable stimulator as programmed using the external device, and/or record information used by the external device for determining the settings of the implantable stimulator. The aggregate data can be collected through multiple users and/or user groups (e.g., clinics and/or specialty networks) of stimulation devices such as implantable stimulators. For example, the aggregate data may be used to generate recommendations for the settings for each individual patient based on settings known to be effective for other patients sharing common and/or similar pathological conditions and device characteristics.

The above detailed description includes references to the accompanying drawings, which form a part of the detailed description. The drawings show, by way of illustration, specific embodiments in which the invention may be practiced. These embodiments are also referred to herein as “examples.” Such examples may include elements in addition to those shown or described. However, the present inventors also contemplate examples in which only those elements shown or described are provided. Moreover, the present inventors also contemplate examples using combinations or permutations of those elements shown or described.

Method examples described herein may be machine or computer-implemented at least in part. Some examples may include a computer-readable medium or machine-readable medium encrypted with instructions operable to configure an electronic device to perform methods as described in the above examples. An implementation of such methods may include code, such as microcode, assembly language code, a higher-level language code, or the like. Such code may include computer readable instructions for performing various methods. The code may form portions of computer program products. Further, in an example, the code may be tangibly stored on one or more volatile, non-transitory, or non-volatile tangible computer-readable media (referred to herein as computer readable medium), such as during execution or at other times. Examples of these tangible computer-readable media may include, but are not limited to, hard disks, removable magnetic disks or cassettes, removable optical disks (e.g., compact disks and digital video disks), memory cards or sticks, random access memories (RAMs), read only memories (ROMs), and the like. The term “machine” may include at least one processor/controller, including one processor/controller to implement all of the instructions, at least two processors/controllers where one processor/controller operates on some of the instructions and other processor(s)/controller(s) operate on other instructions, or at least two processors/controllers where each processor/controller is capable of operating on the same instructions. Thus, for example, distributed systems or systems with shared resources are contemplated.

The above description is intended to be illustrative, and not restrictive. For example, the above-described examples (or one or more aspects thereof) may be used in combination with each other. Other embodiments may be used, such as by one of ordinary skill in the art upon reviewing the above description. The scope of the invention should be determined with reference to the appended claims, along with the full scope of equivalents to which such claims are entitled.

What is claimed is:

1. A method, comprising:
using a neurostimulator to use stimulation parameters to deliver electrical energy to tissue in a patient; and
using processing system to provide a stimulation effects map that maps stimulation effects for different stimulation parameters by:
testing stimulation parameter sets from a plurality of available stimulation parameter sets by controlling the neurostimulator for each of the tested stimulation parameter sets to deliver the electrical energy using the corresponding stimulation parameter set, wherein the plurality of available stimulation parameter sets includes the tested stimulation parameter sets and a plurality of untested stimulation parameter sets;
acquiring clinical effect data indicative of a patient response to the electrical energy delivered to the tissue using at least a first subset of the tested stimulation parameter sets;
acquiring sensed data indicative of a sensed response to the electrical energy delivered to the tissue using at least a second subset of the tested stimulation parameter sets; and
evaluating parameter sets from the plurality of untested stimulation parameter sets to provide evaluated parameter sets, including for each of the evaluated parameter sets, determine an estimated response by estimating at least one of the patient response or the sensed response to the electrical energy using the acquired clinical effect data and the acquired sensed data, wherein the stimulation effects map includes the acquired clinical effect data, the acquired sensed data, and the estimated responses for the evaluated parameter sets.

2. The method of claim 1, further comprising using the processing system to:
choose, based on at least one or more of the estimated responses, a stimulation parameter set from the plurality of available stimulation parameter sets as a chosen stimulation parameter set to be tested; and
control the neurostimulator to deliver electrical energy using the chosen stimulation parameter set.

3. The method of claim 1, wherein the chosen parameter set is selected from one of the evaluated parameter sets, the method further comprising acquiring a tested response, including at least one of clinical effect data or sense data, when the electrical energy is delivered using the chosen parameter set, comparing the acquired tested response to the corresponding one of the estimated responses to provide comparison data, and using the comparison data to update a model used to determine the estimated response.

4. The method of claim 1, wherein the estimated response is determined by interpolating or extrapolating, including by line or surface fittings of the tested stimulation sets.

5. The method of claim 1, wherein the estimated response is determined using knowledge of an expected topology of a search, parameter or other space corresponding to the plurality of available stimulation sets, and the expected topology includes slopes and orientations, peaks, valleys, cliffs or plateaus in the search space.

6. The method of claim 1, further comprising using machine learning for estimating at least one of the patient response or the sensed response to the electrical energy using the acquired clinical effect data and the acquire sensed data.

7. The method of claim 6, wherein the machine learning analyzes data from a current patient, data across multiple patients other than the current patient, or data across multiple patients including the current patient to estimate the at least one of the patient response or the sensed response.

8. The method of claim 1, further comprising at least one of:
sensing an electrical response from the patient to the electrical energy delivered to the tissue, wherein the sensed data is indicative of the sensed electrical response; or
sensing a physical characteristic for the patient, wherein the sensed data is indicative of the physical characteristic for the patient.

9. The method of claim 1, further comprising receiving a user input indicative of the patient response to the electrical energy delivered to the tissue, wherein the user input is indicative of at least one of:
one or more side effects to the electrical energy delivered to the tissue; or
whether and to what extent the electrical energy delivered to the tissue is therapeutically effective.

10. The method of claim 9, wherein the each of the plurality of available stimulation parameter sets includes an electrode configuration and a stimulation waveform configuration.

11. The method of claim 1, wherein both the clinical effect data and the sensed data are associated with at least two stimulation parameters in the plurality of available stimulation parameter sets.

12. The method of claim 11, wherein both the clinical effect data and the sensed data are associated with a stimulation location and a stimulation amplitude.

13. The method of claim 12, wherein both the clinical effect data and the sensed data are further associated with at least one of: a stimulation frequency, a stimulation pulse width, a stimulation location and a stimulation amplitude.

14. The method of claim 1, wherein the sensed data includes features of a sensed signal, the method further comprising determining a difference between the estimated sensed response and the sensed signal, and using the determined difference to choose the stimulation parameter set as the chosen stimulation parameter set to be tested.

15. The method of claim 1, wherein the sensed data includes features of a sensed signal, the method further comprising determining a difference between the sensed signal and the estimated sensed response, determining the estimated patient response based on the determined difference and displaying the estimated patient response on a user interface.

16. A non-transitory machine-readable medium including instructions, which when executed by a machine, cause the machine to perform a method, comprising:
using a neurostimulator to use stimulation parameters to deliver electrical energy to tissue in a patient; and
using processing system to provide a stimulation effects map that maps stimulation effects for different stimulation parameters by:
testing stimulation parameter sets from a plurality of available stimulation parameter sets by controlling the neurostimulator for each of the tested stimulation parameter sets to deliver the electrical energy using the corresponding stimulation parameter set, wherein the plurality of available stimulation parameter sets includes the tested stimulation parameter sets and a plurality of untested stimulation parameter sets;
acquiring clinical effect data indicative of a patient response to the electrical energy delivered to the tissue using at least a first subset of the tested stimulation parameter sets;
acquiring sensed data indicative of a sensed response to the electrical energy delivered to the tissue using at least a second subset of the tested stimulation parameter sets; and
evaluating parameter sets from the plurality of untested stimulation parameter sets to provide evaluated parameter sets, including for each of the evaluated parameter sets, determine an estimated response by estimating at least one of the patient response or the sensed response to the electrical energy using the acquired clinical effect data and the acquired sensed data, wherein the stimulation effects map includes the acquired clinical effect data, the acquired sensed data, and the estimated responses for the evaluated parameter sets.

17. A system, comprising:
a neurostimulator configured to use stimulation parameters to deliver electrical energy to tissue in a patient; and
a processing system configured to provide a stimulation effects map that maps stimulation effects for different stimulation parameters by:
testing stimulation parameter sets from a plurality of available stimulation parameter sets by controlling the neurostimulator for each of the tested stimulation parameter sets to deliver the electrical energy using the corresponding stimulation parameter set, wherein the plurality of available stimulation parameter sets includes the tested stimulation parameter sets and a plurality of untested stimulation parameter sets;
acquiring clinical effect data indicative of a patient response to the electrical energy delivered to the tissue using at least a first subset of the tested stimulation parameter sets;
acquiring sensed data indicative of a sensed response to the electrical energy delivered to the tissue using at least a second subset of the tested stimulation parameter sets; and
evaluating parameter sets from the plurality of untested stimulation parameter sets to provide evaluated parameter sets, including for each of the evaluated parameter sets, determining an estimated response by estimating at least one of the patient response or the sensed response to the electrical energy using the acquired clinical effect data and the acquired sensed data, wherein the stimulation effects map includes the acquired clinical effect data, the acquired sensed data, and the estimated responses for the evaluated parameter sets.

18. The system of claim 17, wherein the processing system is configured to:
based on the stimulation effects map including at least one or more of the estimated responses, choose a stimulation parameter set from the plurality of available stimulation parameter sets as a chosen stimulation parameter set to be tested; and
control the neurostimulator to deliver electrical energy using the chosen stimulation parameter set.

19. The system of claim 17, wherein the processing system is configured to determine the estimated response by interpolating or extrapolating, including by line or surface fittings of the tested stimulation sets.

20. The system of claim 17, wherein the processing system is configured to determine the estimated response using knowledge of an expected topology of a search, parameter or other space corresponding to the plurality of available stimulation sets, and the expected topology includes slopes and orientations, peaks, valleys, cliffs or plateaus in the search space.
